(19) United States
(12) Patent Application Publication
(10) Pub. No.: US 2024/0124316 A1
(43) Pub. Date: Apr. 18, 2024

(54) ZSM-5 zeolites with wood lignin oxidized or not

(71) Applicants: Centre national de la recherche scientifique, Paris (FR); Université de Strasbourg, Strasbourg (FR); Universidade Federal do Rio de Janeiro, Rio de Janeiro (BR)

(72) Inventors: Benoit LOUIS, Geispolsheim (FR); Rogeria Bingre, Strasbourg (FR); Cristina Megias Sayago, Strasbourg (FR); Marcelo Maciel Pereira, Rio de Janeiro (BR); Camila Gomes Flores, Strasbourg (FR)

(21) Appl. No.: 18/254,504

(22) PCT Filed: Nov. 26, 2021
(86) PCT No.: PCT/EP2021/083113

§ 371 Date: May 25, 2023

(30) Foreign Application Priority Data
Nov. 26, 2020 (EP) 20306449.8

(51) Int. Cl. C01B 39/40; B01J 29/40; B01J 35/61; B01J 35/63; B01J 37/00; B01J 37/03; C07C 1/24; C07C 4/06

(52) U.S. Cl. CPC C01B 39/40; B01J 29/40; B01J 35/615; B01J 35/633; B01J 37/0018; B01J 37/03; C07C 1/24; C07C 4/06; B01J 2229/24; C01P 2002/72; C01P 2004/03; C01P 2006/12; C01P 2006/14; C07C 2529/40

(57) ABSTRACT

The present invention relates to a process for preparing a zeolite ZSM-5 presenting a Si/Al molar ratio comprised between 2 and 8, preferably between 3 and 8, comprising the following steps: a) mixing at least one silicon source, at least one aluminum source, at least one organic template and at least one aqueous solvent, in order to obtain a synthesis mixture in solution or gel form; b) ageing the mixture obtained in step a) at a temperature of between 20° C. and 200° C. during at least 30 minutes; and d) crystallizing the resulting mixture during at least 24 hours, wherein a step c) of adding wood lignin or oxidized wood lignin to the mixture is performed after step a) or after step b). It also relates to a zeolite which is obtainable by such a process, and to its use.

The present invention concerns novel ZSM-5 zeolites obtained with lignin and/or oxidized lignin, and their process of preparation.

Zeolites are crystalline aluminosilicates which have a uniform crystal structure characterized by a large number of regular small cavities interconnected by a large number of even smaller channels. It was discovered that, by virtue of this structure consisting of a network of interconnected uniformly sized cavities and channels, crystalline zeolites are able to accept for absorption molecules having sizes below a certain well defined value whilst rejecting molecules of larger size, and for this reason they became to be known as “molecular sieves”. This characteristic structure also confers them catalytic properties, especially for certain types of hydrocarbon conversions, such as Fluid Catalytic Cracking (FCC) or the conversion of methanol into light olefins (MTO).

The ZSM (Zeolite Socony Mobil) family of zeolites is well-known and their preparation and properties have been extensively described. Thus, for example, one type of the ZSM family or zeolites is that known as ZSM-5. ZSM-5 is an aluminosilicate zeolite belonging to the pentasil family of zeolites, and which has the following chemical formula:

NaₙAlₙSi₉₆₋ₙO_{192·}16H₂O(0<n<11)

Based on the unique pore structure of ZSM-5, this zeolite can be applied extensively as a catalyst material in various processes. ZSM-5 zeolite has been shown to be a particularly useful catalyst in reactions involving aromatic compounds. It exhibits unique selectivity in the conversion of olefins, naphtenes, alcohols, ethers and alkanes into aromatics and in reactions such as isomerization, alkylation, dealkylation and transalkylation of aromatics. When ZSM-5 is used in catalytic cracking of petroleum feedstocks (FCC), enhancement of gasoline octane number is achieved. Accordingly, ZSM-5 has been used as an additive to other cracking catalysts, e.g. Y zeolite, to improve gasoline octane number and LPG yields. The use of zeolite as catalysts has seen a great increase in the past decades due to their potential in current and emerging technologies (Corma, A. Chem. Rev. 1997, 97, 2373-2420; Corma, A. and Jones S. Zeolites as catalysts for the synthesis of fine chemicals, in Zeolites and Catalysis, Wiley-VCH Verlag GmbH & Co. KGaA, Wienheim, 2010; Jacobs, P. A.; Dusselier, M.; Sels, B. F. Angew. Chemie—Int. Ed. 2014, 53, 8621-8626; Dusselier, M.; Van Wouwe, P.; Dewaele, A.; Jacobs, P. A.; Sels, B. F. Science. 2015, 349, 78-80; Mintova, S.; Jaber, M.; Valtchev, V. Chem. Soc. Rev. 2015, 44, 7207-7233).

In particular, ZSM-5 type being highly siliceous (Si/Al>10) is currently explored in different techniques such as hydrocarbon cracking, methanol-to-olefins or isomerization, thanks to its structure of narrow pores and channels. However, many scientists have developed strategies to tune the characteristics of this type of zeolite for better catalytic and adsorption performances. For instance, Rimer et al. (Rimer, J. D.; Lobo, R. F.; Vlachos, D. G. Langmuir 2005, 21, 8960-8971) have studied the impact of zeolite growth modifiers on the size and morphology of the crystals. The use of polyamines, proteins and sugars had a strong influence on the assembly of crystals, allowing these to mimic biomineralization processes and thus leading to interesting crystal features.

The efficiency of ZSM-5 zeolites, notably in FCC processes, increases with the aluminum content in the crystalline network: the amount of active sites (acid sites) is proportional to the amount of aluminum.

Recently, Louis et al. (Pereira, M. M.; Games, E. S.; Silva, A. V.; Pinar, A. B.; Willinger, M. G.; Shanmugam, S.; Chizallet, C.; Laugel, G.; Losch, P.; Louis, B. Chem. Sci. 2018, 9, 6532-6539) presented ZSM-5 zeolites exhibiting the lowest Si/Al ratio (being equal to 8+/−0.5), which were obtained using cheap sugarcane residues as crystal growth modifiers.

However, there still exists a need to obtain a ZSM-5 zeolite presenting the lowest amount of silica as possible, and therefore the highest amount of aluminum as possible, in order to improve further its properties, and notably its catalytic activity.

There is also a need for a ZSM-5 zeolite presenting good properties in terms of cationic exchange, and which may be useful in many applications such as detergents, in the remediation of heavy metal-contaminated soils or waters, or in purifying soils or waters from radioactive elements such as cesium.

Besides, there also exists a need for a ZSM-5 zeolite which may be obtained via a simple process, and notably without using fluorides.

The present invention solves this problem: it relates to ZSM-5 zeolites presenting a high amount of aluminum, i.e. a Si/Al molar ratio comprised between 2 and 8, preferably between 3 and 8. They are obtained thanks to the use of wood lignin or an oxidized wood lignin in the process of preparation. Such a process is performed in specific conditions, and confers to the obtained zeolites their interesting properties. These zeolites indeed present a Si/Al molar ratio comprised between 2 and 8, preferably between 3 and 8, and show a very interesting activity in FCC, while maintaining their selectivity towards targeted light olefins such as ethylene and propylene.

Thus, the present invention relates to a process for preparing a zeolite ZSM-5 exhibiting a Si/Al molar ratio comprised between 2 and 8, preferably between 3 and 8, comprising the following steps:
a) mixing at least one silicon source, at least one aluminum source, at least one organic template and at least one aqueous solvent, in order to obtain a synthesis mixture in solution or gel form;
b) ageing the mixture obtained in step a) at a temperature between 20° C. and 200° C. during at least 30 minutes; and
d) crystallizing the resulting mixture during at least 24 hours, wherein a step c) of adding wood lignin or oxidized wood lignin to the mixture is performed after step a) or after step b).

Preferably, according to a first embodiment, the present invention relates to a process for preparing a zeolite ZSM-5 presenting a Si/Al molar ratio comprised between 2 and 8, preferably between 3 and 8, comprising the following steps:
a) mixing at least one silicon source, at least one aluminum source, at least one organic template and at least one aqueous solvent, in order to obtain a synthesis mixture in solution or gel form;
b) ageing the mixture obtained in step a) at a temperature between 20° C. and 200° C. during at least 30 minutes;
c) adding wood lignin or oxidized wood lignin to the mixture of step b); and
d) crystallizing the mixture of step c) during at least 24 hours.

Preferably, according to a second embodiment, the present invention relates to a process for preparing a zeolite ZSM-5 presenting a Si/Al molar ratio comprised between 2 and 8, preferably between 3 and 8, comprising the following steps:
a) mixing at least one silicon source, at least one aluminum source, at least one organic template and at least one aqueous solvent, in order to obtain a synthesis mixture in solution or gel form;
c) adding wood lignin or oxidized wood lignin to the mixture of step a);
b) ageing the mixture obtained in step c) at a temperature between 20° C. and 200° C. during at least 30 minutes; and
d) crystallizing the mixture of step b) during at least 24 hours.

The present invention also relates to a zeolite ZSM-5 presenting a Si/Al molar ratio comprised between 2 and 8, preferably between 3 and 8, which is obtainable by the process according to the invention.

The present invention also relates to the use of such a zeolite in hydrocarbons conversion reactions, preferably Fluid Catalytic Cracking (FCC).

The process for preparing a zeolite ZSM-5 presenting a Si/Al molar ratio comprised between 2 and 8, preferably between 3 and 8, of the invention, comprises the following steps:
a) mixing at least one silicon source, at least one aluminum source, at least one organic template and at least one aqueous solvent, in order to obtain a synthesis mixture in solution or gel form;
b) ageing the mixture obtained in step a) at a temperature between 20° C. and 200° C. during at least 30 minutes; and
d) crystallizing the resulting mixture during at least 24 hours, wherein a step c) of adding wood lignin or oxidized wood lignin to the mixture is performed after step a) or after step b).

The process of the invention comprises two embodiments:
In the first one, the step of adding wood lignin or oxidized wood lignin to the mixture (step c) is performed after the ageing step (step b).
In the second one, the step of adding wood lignin or oxidized wood lignin to the mixture (step c) is performed after the initial mixing step (step a) and before the ageing step (step b).

The process may further comprise a step e) of separating the solid obtained in step d) by means of centrifugation, filtration or evaporation of the solvent. Typically the separation step e) may be performed by filtration of the solid obtained in step d) on a membrane, such as a nylon membrane.

At the end, the process may also further comprise a drying and/or calcination step f), which preferably occurs after step e). Said drying and/or calcination step f) typically comprises a drying step under vacuum, and a calcination under air, preferably at a temperature between 500° C. and 600° C., typically for at least some hours (for example at least 2 hours, preferably at least 10 hours, preferably 15 hours).

Preferably, the process according to the invention comprises, between steps c) and d), a doping step. When the process of the invention is according to the first embodiment, then the doping step may be performed after oxidation (step c) and before crystallization (step d).

When the process of the invention is according to the second embodiment, then the doping step may be performed after ageing (step b) and before crystallization (step d).

The doping step may be made by adding a metal cation in the synthesis recipe, in order to introduce a metal function. Said metal function might be useful for bifunctional catalysis application. For example, the doping step may be performed by adding cations such as cations of transition metals, and for example iron, cobalt, copper, nickel, platinum or palladium cations.

Step a) comprises mixing at least one silicon source, at least one aluminum source, at least one organic template and at least one aqueous solvent, in order to obtain a synthesis mixture in solution or gel form.

The silicon source may be chosen from tetraethylorthosilicate (TEOS) (C₈H₂₀O₄Si), colloidal silica, disodium metasilicate (Na₂O₃Si) and their mixtures. Preferably, the silicon source is tetraethylorthosilicate (TEOS) (C₈H₂₀O₄Si).

The aluminum source may be chosen from sodium aluminate (NaAlO₂), aluminum isopropoxide (C₉H₂₁AlO₃), aluminum sulfate (Al₂O₁₂S₃) and their mixtures. Preferably, the aluminum source is sodium aluminate (NaAlO₂).

The organic template may be chosen from tetrapropyl ammonium hydroxide (TPAOH) (C₁₂H₂₉NO), tetramethyl ammonium hydroxide (TMAOH) (C₄H₁₃NO), tetramethyl ammonium bromide (C₄H₁₂BrN), tetrapropyl ammonium bromide (C₁₂H₂₈BrN) and their mixtures. Preferably, the organic template is tetrapropyl ammonium hydroxide (TPAOH) (C₁₂H₂₉NO).

Preferably, the aqueous solvent is water.

Salts may also be present in the mixture, such as, for example sodium chloride. Said salts may provide more ionic strength to the synthesis mixture obtained in step a), and may bring more positive charges to compensate the numerous [AlO₄]⁻ charges.

The Na/Al molar ratio may be adjusted in order to obtain a typical value of around 4 to 15.

In theory, one may further increase the Na/Al molar ratio; however, as zeolites are metastable structures, one may possibly shift to another zeolite structure (FAU, GIS, ANA). Thus salts may be added, but in an amount such that the Na/Al molar ratio is of around 4 to 15, preferably from 4 to 13, preferably from 4 to 6, preferably from 4 to 5.3.

In step a), the silicon source, the aluminum source and the organic template are mixed in the aqueous solvent. Preferably, they are mixed at a temperature of 20-25° C. (i.e. room temperature).

Preferably, step a) comprises the following sub-steps:
a1) mixing at least one aluminum source with at least one organic template in at least one aqueous solvent, in order to obtain a mixture;
a2) adding at least one silicon source, and preferably at least one salt, more preferably at least sodium chloride, into the mixture of step a1), in order to obtain the synthesis mixture in solution or gel form.

Preferably, the molar ratio of the silicon source to the salt, preferably sodium chloride, is of at least 2.20, preferably between 2.20 and 3, preferably between 2.20 and 2.80, preferably between 2.20 and 2.50. Preferably, the molar ratio of TEOS to NaCl is of at least 2.20, preferably between 2.20 and 3, preferably between 2.20 and 2.80, preferably between 2.20 and 2.50.

Then, when the process of the invention is according to the first embodiment, the mixture of step a) is put under ageing, at a temperature between 20° C. and 200° C. during at least 30 minutes; this is step b). When the process of the invention is according to the second embodiment, the oxidized mixture is put under ageing; this is the same step b) (i.e. at a temperature between 20° C. and 200° C. during at least 30 minutes).

Preferably, the temperature of step b) is between 70° C. and 200° C.

Typically, the ageing of step b) is performed during at least 1h, preferably at least 1 h30, preferably between 1 h and 5 h.

Preferably, the ageing of step b) is performed during a time period between 30 minutes and 4 hours, preferably between 1 hour and 3 hours.

The ageing of step b) may comprise a stirring step.

When the process of the invention is according to the first embodiment, after ageing, wood lignin or oxidized wood lignin is added to the mixture of step b); this is step c). When the process of the invention is according to the second embodiment, said step c) of adding wood lignin or oxidized wood lignin to the mixture is performed after the initial mixing (step a), and before ageing (step b).

Lignin is a class of complex organic biopolymers that form key structural materials in the support of tissues of vascular plants. They are abundantly found in wood and bark, and their chemical structure is formed by cross-linked phenolic polymers. The lignin used in the invention (called “wood lignin” by simplicity) originates from wood, bark or from different nutshells, such as walnut shells.

Preferably, the wood lignin used in the invention presents the following fragment of a macromolecule structure with different linkages (Y. Song et al., Green Chem. 21 (2019) 3940), as disclosed in formula (I):

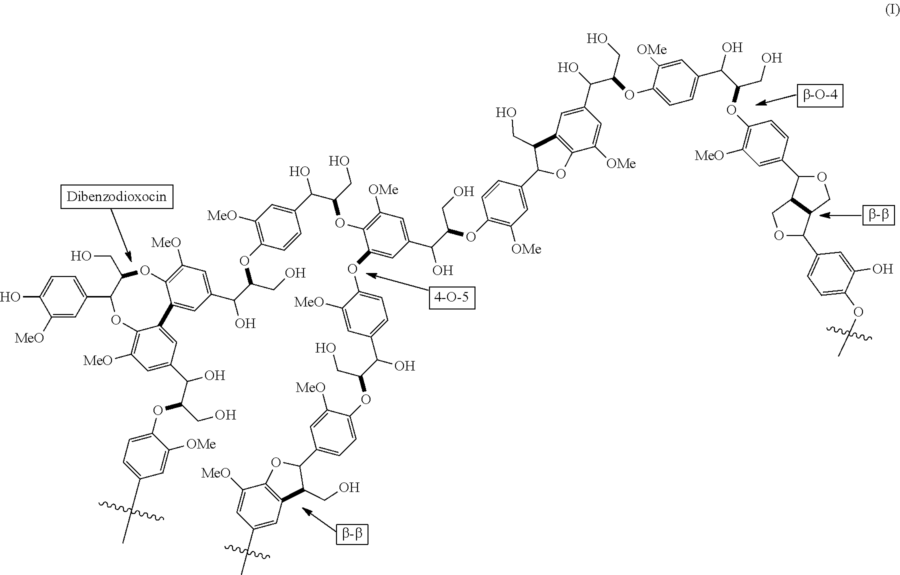

The random organization of hydrocarbons produces a hydrophobic assembly, which may influence the zeolite crystallization during the hydrothermal treatment. The wood lignin used in the present invention may be used as such or may be hydrolyzed. For example, it may be hydrolyzed by mixing the wood lignin with strong alkaline or acidic media. Strong alkaline media include aqueous solutions of strong bases, such as sodium hydroxide or potassium hydroxide. Strong acidic media include aqueous solutions of strong acids, such as sulfuric acid or nitric acid.

The wood lignin used in the invention may be used as such, or in oxidized form.

Preferably the oxidized form of wood lignin according to the invention comprises muconic acid structures of formula (II):

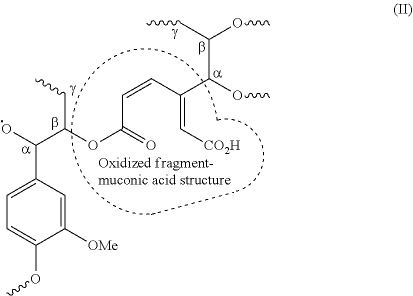

It has to be noted that lignin is typically different from sugar bagasse which contains essentially cellulose and hemi-cellulose.

Lignin constitutes up to 30% of the weight and 40% of the energy content of lignocellulosic biomass (e.g., wood) with the remainder of the biomass being cellulose and hemicellulose. Lignin suitable for use in the present invention can be obtained from the lignocellulosic biomass using any suitable methodology. In an embodiment the lignin is usually having an average molecular weight ranging from between 350 Da and 1900 Da (P. S. Marathe et al., Appl. Energy 236 (2019) 1125-1137).

Preferably, the wood lignin according to the present invention presents a sulfur atomic content of between 0.8 at % to 8 at %, preferably from 3 at % to 6 at % by weight of the total weight of the dry wood lignin.

Preferably, the wood lignin according to the present invention presents a carbon atomic content of between 35 at % to 55 at %, preferably from 37 at % to 51 at % by weight of the total weight of the dry wood lignin.

Preferably, the wood lignin according to the present invention presents a hydrogen atomic content of between 3.8 at % to 6.5 at %, preferably from 4 at % to 6.2 at % by weight of the total weight of the dry wood lignin.

The wood lignin according to the present invention may comprise a nitrogen atomic content of between 0.1 at % to 0.5 at % by weight of the total weight of the dry wood lignin.

Preferably, the wood lignin according to the present invention presents an ash content of between 3% to 10%, preferably from 5% to 8% by weight of the total weight of the dry wood lignin.

The ash content can be defined as the gravimetrically determined residue after ignition at a temperature of 525±25° C., in a wood lignin sample, in percent (weight/weight dry matter of wood lignin sample). In the determination, a sample of wood lignin is weighed in a heat-resistant crucible, dried at 105±2° C., and ignited in a muffle furnace at 525±25° C. The ash content is then determined, on a moisture-free basis, from the weight of residues after ignition and the moisture content of the sample.

Preferably, the wood lignin according to the present invention presents a residual carbohydrate content between 5% to 20%, preferably from 8% to 20%, preferably from 10% to 15% by weight of the total weight of the wood lignin.

The carbohydrate content can be defined as the sum of the amounts of the five principal, neutral monosaccharides : arabinose, galactose, glucose, mannose and xylose in anhydrous form, in a sample, in milligrams per gram. In the determination, the samples are hydrolyzed with sulfuric acid using a two-step technique. The amounts of the different monosaccharides are determined using ion chromatography.

Preferably, some part of the wood lignin according to the present invention may be extracted in at least one organic solvent. Said organic solvent may be polar or apolar. Preferably, some part of the wood lignin according to the present invention is extracted in at least one polar organic solvent, and some other part is extracted in at least one apolar organic solvent. Preferably, the organic solvent is chosen from hexane, chloroform and acetone.

Preferably, the extraction is performed in a Soxhlet under solvent reflux conditions for at least 1 h, preferably for at least 2 h, preferably between 2 and 6 h.

Liquid chromatography coupled with mass spectrometry analyses of the extracted components composition typically shows the presence of fatty (C₁₆-C₂₄) acids as well as dehydroabietic and abietic acids acids (present in resin) and sterol components (campasterol, sistosterol, sitostanol and cholesterol).

Preferably, an amount of 3% to 10% by weight of the total weight of the wood lignin is extracted in hexane, preferably an amount of 4% to 8% by weight.

Preferably, an amount of 3% to 10% by weight of the total weight of the wood lignin is extracted in chloroform, preferably an amount of 4% to 8% by weight.

Preferably, an amount of 1% to 10% by weight of the total weight of the wood lignin is extracted in acetone, preferably an amount of 1.5% to 5% by weight.

Preferably, the wood lignin according to the present invention presents a Klason lignin content between 50% to 95%, preferably from 50% to 90%, preferably from 50% to 80%, preferably from 60% to 70% by weight of the total weight of the wood lignin.

In order to measure the Klason lignin content, the cellulose is first partially depolymerized into oligomers by keeping the wood lignin sample in 72% sulfuric acid at 30° C. for 1 h. Then, the acid is diluted to 4% by adding water, and the depolymerization is completed by either boiling (100° C.) for 4 h or pressure cooking at 2 bar (124° C.) for 1 h. The acid is washed out and the sample dried. The residue that remains is termed Klason lignin.

Typically, the oxidized wood lignin is obtained from wood lignin by chemical treatment. Preferably, the chemical treatment is chosen from an alkali treatment, a treatment with molecular oxygen and a treatment with hydrogen peroxide.

Typically, the oxidation is performed by mixing the wood lignin with an alkali solution. Said alkali solution may be any conventional solution of a strong base. The strong base may be chosen among hydroxides of the alkali metals and alkaline earth metals. For example, the alkali solution is a sodium hydroxide solution or a potassium hydroxide solution. The base is typically used at a high concentration, preferably from 0.1 M to 1 M, more preferably from 0.15 M to 0.5 M. The mixture of the wood lignin with the alkali solution is preferably heated, typically at a temperature between 70° C. and 100° C., preferably of 80° C. to 90° C., typically for at least 1 h. The duration influences the global oxidation of the product. Then the mixture is preferably cooled to room temperature. Optionally the solvent is evaporated, and oxidized wood lignin is thus obtained.

Another chemical treatment for oxidizing wood lignin uses molecular oxygen and a subsequent treatment with a mixture of formic acid and a formate salt, such as sodium formate. Said subsequent treatment is preferably heated, typically at a temperature of 90° C. to 150° C., preferably of 100° C. to 120° C., typically for at least a few hours, preferably at least 20 h. Finally, an extraction is performed with an organic solvent such as ethyl acetate, and the soluble fraction is collected. It comprises aromatic compounds of low molecular weight, which are soluble in the organic solvent.

Another chemical treatment for oxidizing wood lignin uses hydrogen peroxide (H₂O₂). Typically said treatment includes mixing wood lignin with a solution containing H₂O₂ (for example from 1 to 3.3 M) and H₂SO₄ (for example from 0.5 to 1.5 M) at a temperature between 20° C. and 90° C., preferably 80° C. to 90° C. Preferably, 3 M H₂O₂ and 1.2 M H₂SO₄ concentrations are used at a preferred temperature of 80° C. to 90° C. The duration of the oxidation protocol ranges between 2 h and 4 h, preferably between 1.5 h and 2 h. Then, the main part of the reaction products remain in an insoluble fraction, considered as the oxidized lignin which may be filtered on a Nylon membrane. The yield is preferably comprised between 70% and 90%, preferably between 75% and 85%. The acidic filtrate contains the acid-soluble part of the lignin (yield between 15% and 25%).

Preferably, the oxidized wood lignin according to the present invention presents a carbon atomic content of between 50 at % to 60 at %, preferably from 52 at % to 58 at % by weight of the total weight of the dry wood lignin.

Preferably, the oxidized wood lignin according to the present invention presents a hydrogen atomic content of between 4.5 at % to 6 at %, preferably from 4.7 at % to 5.5 at % by weight of the total weight of the dry wood lignin.

Preferably, in step c), the wood lignin or oxidized wood lignin is added to the mixture of step b) in an amount ranging from 0.1 to 0.8 g. The mass ratio of wood lignin (or oxidized wood lignin) to the aluminum source (Al) was varied between 0.8 to 15, preferably between 2 and 15, more preferably between 3.8 and 15, expressed in weight of lignin by weight of aluminum source in the synthesis recipe.

Finally, the process of the invention comprises crystallizing the resulting mixture during at least 24 hours; this is step d). According to the process of the first embodiment, the mixture obtained after addition of wood lignin or oxidized wood lignin (step c) is crystallized. According to the process of the second embodiment, the mixture obtained after ageing (step b) is crystallized.

Preferably, the crystallization is performed during a time period of between 24 and 72 hours, preferably between 48 hours and 72 hours.

Preferably, the crystallization is performed at a temperature of between 100° C. and 200° C., preferably of between 150° C. and 190° C.

The ZSM-5 zeolite which is obtainable by the process of the invention, also called “ZSM-5 zeolite according to the invention”, presents a Si/Al molar ratio comprised between 2 and 8, preferably between 3 and 8, preferably presents a Si/Al molar ratio comprised between 3 and 7.6, preferably between 3 and 7, preferably between 3 and 5, preferably between 3 and 4.

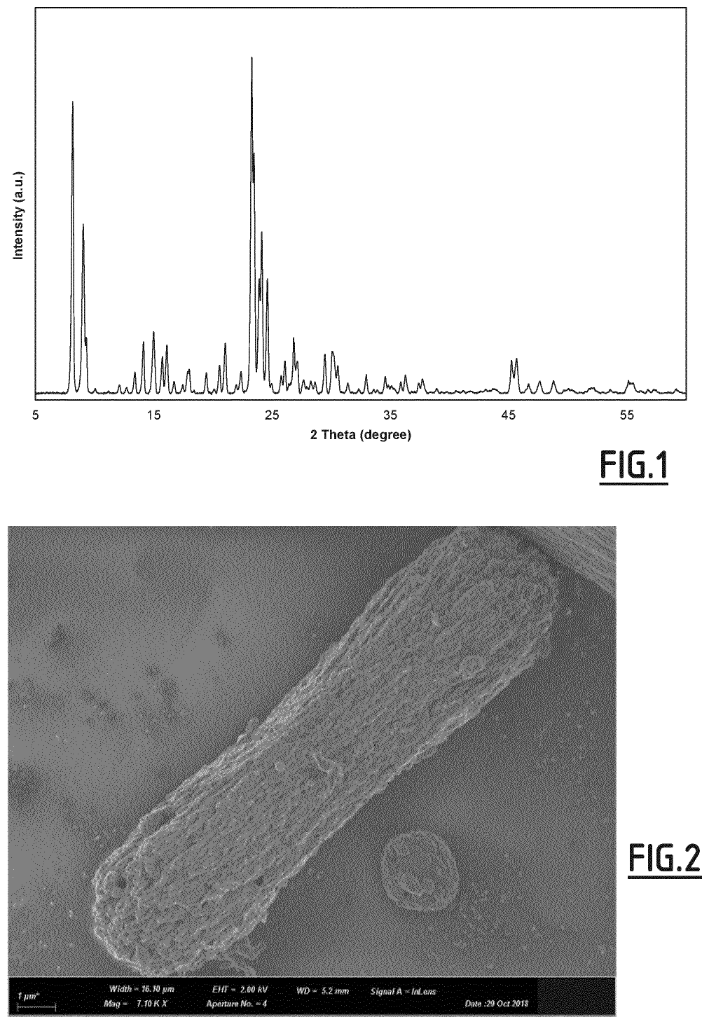

The purity of the crystalline structure of the ZSM-5 zeolite of the invention may be assessed by X-ray diffraction (XRD) analysis. For example, the pattern of the z_500LO catalyst is reported hereunder as an example. All samples exhibit the sole presence of the MFI crystalline structure, characteristic from the ZSM-5 zeolite. FIG. 1 represents the XRD pattern of z_500LO sample prepared using oxidized lignin (prepared as explained in the example below).

Besides, preferably, the ZSM-5 zeolite of the invention presents a microporous structure. By “microporous structure,” it is meant that the ZSM-5 zeolite presents:
a specific surface area (S_{BET}) of between 150 m²/g and 250 m²/g, preferably between 180 m²/g and 220 m₂/g, and
a total pore volume of between 0.01 cm³/g and 0.5 cm³/g, preferably between 0.05 cm³/g and 0.2 cm³/g, wherein the microporous volume is around 70% of the total pore volume, as measured by N₂ adsorption-desorption.

Preferably, the ZSM-5 zeolite of the invention is in the form of crystals presenting the shape of a “peanut”. By “shape of a “peanut””, it is meant an oblong shape, with a length and a width, the length being greater than the width, for example at least 2 times greater, preferably at least 3 times greater. This is notably shown in FIG. 2. Preferably, these crystals have a length of from 10 μm to 30 μm, preferably of from 15 μm to 25 μm.

The present invention also relates to the use of a ZSM-5 zeolite according to the invention as a catalyst. Particularly the present invention also relates to the use of such a ZSM-5 zeolite in the conversion of hydrocarbons. Such conversions include converting high molecular weight hydrocarbon fractions of petroleum crude oils into gasoline and light olefin gases (such as C₂-C₄ olefins). Preferably, the hydrocarbon conversion is FCC.

Preferably, the ZSM-5 zeolite according to the invention is used for converting methanol into olefins (MTO).

Preferably, according to another embodiment, the ZSM-5 zeolite according to the invention is used for cracking n-hexane.

The present invention also relates to a process for converting high molecular weight hydrocarbon fractions of petroleum crude oils into gasoline and light olefin gases, which comprises the step of mixing high molecular weight hydrocarbon fractions of petroleum crude oils with a ZSM-5 zeolite according to the invention. The present invention also relates to a process for converting methanol to olefins, which comprises the step of reacting methanol over a ZSM-5 zeolite according to the invention.

The present invention also relates to a process for cracking n-hexane, which comprises the step of reacting n-hexane over a ZSM-5 zeolite according to the invention.

In these processes, the ZSM-5 zeolite of the invention is preferably activated prior to use in a reaction. The activation may be performed in conventional manners, typically by heating, for example under nitrogen at 500° C. for 2 h.

Then, the conversion or cracking process may occur under classical conditions, known in the art.

The present invention also relates to the use of a ZSM-5 zeolite according to the invention in any one of the following applications:
for adsorbing or desorbing liquids and/or gases. This may be useful in industrial treatments, such as water treatment or industrial gases treatment;
for selective separations of gas or liquid mixtures, i.e. as molecular sieves;
as detergents;
for treating pesticides, organic chlorine or hydrocarbons-loaded effluents;
for remediating heavy metals contained in soils or waters. Said heavy metal may for example be any toxic heavy metal, such as arsenic, mercury, lead, cadmium or chromium;
for purifying soils or waters from radioactive elements, such as cesium.

The present invention also relates to the use of a ZSM-5 zeolite according to the invention as a seed in an industrial process for preparing ZSM-5 zeolites presenting a Si/Al molar ratio comprised between 2 and 8, preferably between 3 and 8. Indeed, the ZSM-5 zeolite according to the invention may be added in a small amount (typically 1% to 5% by weight) in an industrial conventional process for preparing ZSM-5 zeolites, known in the art, in order to produce large-scale zeolites with said Si/Al molar ratio comprised between 2 and 8, preferably between 3 and 8.

More specifically, typically, the present invention also relates to a process for preparing large-scale ZSM-5 zeolites presenting a Si/Al molar ratio comprised between 2 and 8, preferably between 3 and 8, comprising introducing a small amount, typically 1% to 5% by weight, of a ZSM-5 zeolite according to the invention, into a mixture of conventional ingredients used for preparing ZSM-5 zeolites.

The present invention is now illustrated by the following example, which is given as illustrative purpose only.

### Example 1: Synthesis of ZSM-5 Zeolite Incorporating Wood Lignin

### Lignins

Several batch of lignins have been tested: alkali lignin (low sulfonate content, Sigma Aldrich: 46.5 at % C and 4.9 at % H) (also called kraft alkali lignin), wood lignin (from the Kirov plant, city of Kirov, Russia, hereafter “wood lignin”), oxidized wood lignin and walnut shells (ecoshell).

However, the wood lignin with the following composition (Table 1) was selected to produce high Al-containing ZSM-5 zeolite:

TABLE 1
Composition of wood lignin sample

| | Residual | Extractive substances, % | | | | |
|---|---|---|---|---|---|---|
| Ash | carbohydrate | | | Chlo- | | Klason |
| content, | content, | Full | Hexane | roform | Acetone | lignin, |
| % | % | content | extract | extract | extract | % |
| 6.9 | 13.5 | 14.3 | 5.9 | 6.0 | 2.4 | 65.3 |

This wood lignin was dried at room temperature and sieved until particle size was of 0.5 mm.

Detailed Analysis of the Wood Lignin (i.e. From the Kirov Plant, City of Kirov, Russia as Mentioned Before)

### Extractive Substances Analysis

Lignin extraction was performed in Soxhlet extractor in series of n-hexane, chloroform and acetone extractions. The extracted substances were dried on a rotary vacuum evaporator at a temperature 40° C.

The obtained extracted substances were analyzed by GS-MS (Agilent G 1530A in tandem with mass selective detector Agilent HP 5973, capillary column HP-5 25 m×0.2 mm with a liquid phase of 5% phenylmethylsiloxane).

### The Relative Component Composition of Wood Lignin Extracted Substances

Analysis of the extracted components composition showed the presence of fatty (C₁₆-C₂₄) acids and resin acids (dehydroabietic and abietic acids; but also 7-oxodehydroabietic acid methyl ester) and sterol components (campasterol, sistosterol, sitostanol but also cholesterol).

### Elementary Composition Analysis

Analysis of wood lignin elementary composition was performed on the machine “elementar vario EL”.

The results are shown in the table below.

TABLE 2
Elemental composition of hydrolysis lignin O

| C | H | O | N | S |
|---|---|---|---|---|
| 47.24 | 4.34 | 30.72 | 0.15 | 0.87 |

### Functional Group Analysis

Content of methoxy groups in wood lignin was determined by Zeisel—Vieböck—Schwappach method (G. Zakis, Functional Analysis of Lignins and Their Derivatives, 1994). Hydroxy groups content was determined by methylation with dimethylsulfate followed by methoxyl groups analysis.

The results are shown in table 3.

TABLE 3
Functional group content in hydrolysis lignin

| Methoxyl groups, % | Hydroxyl groups, % |
|---|---|
| 10.64 | 5.84 |

### FTIR Analyses

FTIR analyses of wood lignin were carried out in a reflectance mode using a Nicolet™ iS™ 50 FT-IR Spectrometer (Thermo Nicolet Corp. Madison, WI, USA) equipped with a build in diamond ATR unit. The region between 4000 and 400 cm⁻¹ with a resolution of 4 cm⁻¹ and 66 scans was recorded (data not shown).

The characteristic vibration bands of lignin complex matter corresponding to C—H deformation in guaiacyl units, aromatics stretching were observed at 1182 cm⁻¹ and 1602 cm⁻¹, respectively.

### Preparation of Oxidized Lignin

The oxidation procedure of the wood lignin of Table 1 was performed as follows:
i) At first, dissolution of wood lignin in a round-bottomed 3-neck flask with a volume of 1 L, equipped with a thermometer, a propeller stirrer and a reflux condenser.
ii) The alkali solution (3.6 g of NaOH in 0.5 L of water, thus 0.18 M) is then placed in the flask, and, through the side-neck, small portions of lignin in amount of 20 g are added under vigorous stirring. The temperature in the flask is preferably raised to 85° C. let under stirring for at least 1 h.
iii) The solution is cooled to room temperature and approximately 0.5 L of solution of oxidized lignin is obtained at a concentration of 40 g/L.
iv) The solvent is then evaporated and the dark brown solid recovered.

### Detailed Analysis of the Oxidized Wood Lignin Thus Obtained

### ¹³C NMR Analysis

NMR-analysis of wood lignin was performed on a spectrometer Bruker MSL—400. Frequency 100.6 MHz, 7 mm zirconium rotor rotates at a frequency of 8 kHz, the pulse width of H¹ and C¹³ was 90°, pulse delay of 4 s, contact time 1.5 ms, the number of pulses being 5000.

Solid-state ¹³C NMR analysis allows to quantify structural units contained in lignin (C_{Ar-O}, C_{Ar-C}, C_{Ar-H}) (E. A. Capanema, M. Y. Balakshin, J. F. Kadla, A comprehensive approach for quantitative lignin characterization by NMR Spectroscopy. J. Agric. Food Chem. 2004, 52, 1850).

The relative content of carbon atoms with various substituents in phenylpropane units of lignin was estimated in relation to 6 carbon atoms of aryl structure. The results are shown in the table 4 below.

TABLE 4
Quantitative estimation of structural elements in hydrolyzed lignin as
revealed by ¹³C NMR in relation to 6 carbon atoms of aryl structure

| | Structural | Integrated | |
|---|---|---|---|
| Fragment structure | element | region, ppm | Relative content |
| 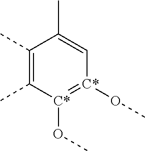 | «C_{Ar-O}» | 162-136 | 1.8 |
| 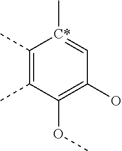 | «C_{Ar-C}» | 136-125 | 1.9 |
| 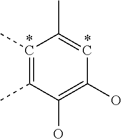 | «C_{Ar-H}» | 125-106 | 2.3 |

### Preparation of the ZSM-5 Zeolites

Three types of ZSM-5 zeolites were prepared.

For this purpose, 0.12 g of NaAlO2 was mixed with 17.7 mL of tetrapropylammonium hydroxide (TPAOH) in 41 mL of distilled water. Then, 0.353 g of NaCl and 3.1 mL of tetraethylorthosilicate (TEOS) were dissolved in the previous solution. Ageing was performed during 2 h at room temperature.

Thereafter, different amounts of the oxidized lignin (obtained as described above) were added, and crystallization occurred during 48 h at 170° C.

The amounts of oxidized lignin are: 100 mg, 300 mg or 500 mg.

Thus, respectively, the corresponding zeolites are called z_100LO, z_300L0 and z_500LO.

It is noticed that during the preparation of the synthesis gel, oxidized lignin was added after mixing all the other reactants. It is important to mention that even at low mass (100 mg), this compound only partially dissolved in the solution.

### Structural Properties

The diffraction patterns of the samples prepared with different quantities of oxidized lignin after calcination step exhibited the sole presence of MFI structure, which indicates the total combustion of the biomass. Moreover, the addition of oxidized lignin did neither lead to a loss in crystallinity nor contribute to the appearance of a new crystalline phase. N₂ adsorption-desorption measurements reported typical type I isotherm related to microporous materials for the three samples. Specific surface areas (S_{BET}) of around 200 m²/g and total pore volume of around 0.1 cm³/g was obtained, where the microporous volume is 70% of the latter (Table 5), confirming that z_xLO possess predominantly a microporous structure. In stark contrast, Gomes et al (Microporous and Mesoporous Mater. 254 (2017) 28-36), reported a type IV isotherm, thus indicating the presence of mesopores in the zeolite while using wood lignin in the synthesis of ZSM-5 zeolite. The pore distribution profiles obtained by the BJH method (i.e. Barrett-Joyner-Halenda method, which is a standard method for measuring pore volume and pore size distribution of solid materials) further confirm the sole presence of micropores in z_xLO materials.

TABLE 5
Specific surface area (S_{BET}), external surface area
(Sₑₓₜ), total pore volume (Vₚₒᵣₑ) and microporous volume (V_{micro})
obtained by N₂ adsorption-desorption

| Sample | S_{BET} (m²/g) | Sₑₓₜ (m²/g) | Vₚₒᵣₑ (cm³/g) | V_{micro} (cm³/g) |
|---|---|---|---|---|
| z_100LO | 200 | 55.9 | 0.108 | 0.071 |
| z_300LO | 201 | 60.1 | 0.105 | 0.069 |
| z_500LO | 195 | 54.7 | 0.102 | 0.069 |

The microstructure of the samples was analyzed by SEM.

z_100LO exhibits the characteristic coffin-shaped crystals associated to ZSM-5 zeolite type. Interestingly, the addition of oxidized lignin leads to the formation of crystals of dimensions around 20 μm presenting the oblong shape of a “peanut”. The increase of the biomass quantity increases also the appearance of these peculiar crystals, until a homogeneity is observed for z_500LO. Observing in more details, these “peanuts” are nothing more than an agglomeration of rectangular filaments that consists in nanocrystals with the shape of “French fries”. It seems that these filaments grow from a central point of the agglomerate that can be the starting matter for those elongated crystals growth. Once again, the same was observed for Si/Al=8 of Pereira et al. (“ZSM-5 SAR8” of Biomass-mediated ZSM-5 zeolite synthesis: when self-assembly allows to cross the Si/Al lower limit, Chemical Science, 2018, 9, 6532-6539), although the crystals exhibited a spherical form. Gomes et al. (Microporous and Mesoporous Mater. 254 (2017) 28-36) also reported a different crystals morphology with high surface roughness formed by the assembly of sub-units which allowed to produce a ZSM-5 material with mesopores (4 nm) and exhibiting a high specific surface area of 450 m²/g.

The mapping of the elements of z_500LO was also performed by EDX coupled to SEM (not represented), and it was detected the presence of sole Si, Al and O elements, confirming the previous assumption of total removal of oxidized lignin. Surprisingly, it was determined a Si/Al ratio of 4, being the lowest ever reported for the ZSM-5 zeolite. Likewise, it was determined that z_100LO has a Si/Al ratio of 8, and z_300L0 a Si/Al ratio of 6.

FIG. 2 represents a SEM image of z_500LO zeolite crystals, which exhibit an unusual oblong shape, named “peanut”-shape morphology.

In order to confirm these results, solid-state NMR was performed. This technique relies on the detection of relevant basic nuclei on the zeolite framework by their natural isotopes (natural abundance in parentheses): ²⁹Si (4.7%), ²⁷Al (>99.9%), and ¹⁷O (0.04%). The resonance lined obtained for ²⁹Si are usually narrow and, due to their important role as framework element (besides ²⁷Al), these nuclei have been widely used in solid-state NMR studies of micro- and mesoporous materials for structural investigations. The most important application of ²⁹Si NMR is due to the relationship between the ²⁹Si chemical shift sensitivity and the degree of condensation of the Si—O tetrahedra, that is the number and type of tetrahedrally coordinated atoms connected to a given SiO₄ unit Si(nAl), with n=0, 1, 2, 3 or 4. The chemical shift ranges from −80 to −115 ppm, with the high-field shift signal for Si(0Al). Here, n indicates the number of Al atoms sharing oxygens with the SiO₄ tetrahedron under consideration.

Differences in the chemical shift between Si(nAl) and Si(n+1Al) are about 5-6 ppm. In this way, the spectra obtained can be used to calculate the framework Si/Al ratio from the NMR signal intensities (I) according to equation 1.

$${( \frac{Si}{Al} )}_{NMR} = \frac{{\sum }_{n = 0}^{4} {I}_{Si ( nAl )}}{{\sum }_{n = 0}^{4} 0.25 {nI}_{Si ( nAl )}} = \frac{0.1 + 0.05 + 0.2 + 0.55 + 0.1}{0.2 \times ( 4 \times 0.1 + 3 \times 0.05 + 2 \times 0.2 + 0.55 )} = 2.67 = 3 \qquad ( 1 )$$

This seems to imply that the presence of oxidized lignin allows the stabilization of the aluminosilicate structure, allowing the lowest Si/Al ratio (SAR) ever reported.

²⁷Al NMR spectra reveal a small existence of extra-framework Al (about 0 ppm) besides the lattice aluminum (tetrahedrally coordinated Al at about 40-65 ppm), which can be negligible and admit a real Si/Al ratio of 3 in the z_500LO framework.

Brønsted and Lewis acid sites of z_500LO were discriminately measured by FTIR of adsorbed pyridine. The spectrum is similar to the one exhibited by conventional samples with bands at the same wavenumber. By integration of the peaks at 1544 and 1455 cm⁻¹ after desorption at 150° C., it was obtained 285 and 14.6 μmol/g for Brønsted and Lewis acid sites, respectively. Moreover, the Al concentration was determined to be 1444 μmol/g. Normally, the total concentration of acid sites (Brønsted+Lewis) should be equal to the concentration of Al in the zeolites. Unfortunately, this is not the case. It might have been an error in Al quantification or a non-negligible fraction of acid sites that are not accessible to pyridine. Nonetheless, high Al content in the aluminosilicate structure directly increases the Bronsted acidity, while Al zoning or extra-framework aluminum species (EFAl) is related to Lewis acid sites. The low value of the latter seems to indicate the nearly absence of these species, as verified by ²⁷Al MAS NMR.

OH DRIFT spectrum of z_500LO showed three bands at: (i) 3745 cm⁻¹ characteristic of isolated Si—OH with very low intensity compared with commercial zeolites; (ii) 3670 cm⁻¹ characteristic of extra-framework aluminum Al—OH of equally low intensity; and (iii) 3620 cm⁻¹ characteristic of zeolite framework with high intensity. Once again, these results suggest the presence of very small quantity of EFAl species.

### Catalytic Performance in the Methanol-to-Hydrocarbons Reaction

In the previous section, it was presented the in depth characterization of z_500LO. It is expected that such low Si/Al ratio may lead to interesting catalytic performance in the MTH reaction. The catalyst lifetime was compared to the commercial CBV3020E (Zeolyst company), along with the coke analysis.

The sample z_500LO exhibited lower capacity to maintain the full conversion of methanol and dimethyl ether than CBV3020E, however its deactivation rate showed to be slower. This may be attributed to the crystal size of the two samples. The commercial zeolite exhibits nanocrystals, which induces shorter diffusion paths for the exit of the reactant/products molecules being able to keep the active sites clean for a longer time. On the other side, once the coke precursors start to poison those sites, the conversion quickly diminishes. In the case of z_500LO, the crystals exhibited an oblong micrometric size, hindering the exit of molecules that slowly deactivate the catalyst. This may be confirmed by the coke analysis that showed lower coke content at 600° C. than CBV3020E. Indeed, this temperature seems non-sufficient to remove all the coke present in z_500LO as there is still a decrease in the mass up to the end of the experiment. This is related to the size of the molecules, which suggest that as bigger as the molecules are, more energy is needed to provide to decompose them. By staying for a longer time in contact with the active sites, coke precursors are able to further react.

Regarding the products selectivity (Table 6), z_500LO exhibited higher olefins selectivity than CBV3020E, especially towards ethylene and butylene isomers. On the other side, there was a similar formation of compounds with 5 carbons or more for both samples. This is a surprising result as higher aluminum content is associated with higher active sites and, consequently, higher selectivity towards heavier products. It seems that the crystals morphology plays here an important role in the catalytic performance beside the acidity.

TABLE 6
Selectivity in ethylene (C₂H₄), propylene (C₃H₆), butylene
isomers (C₄H₈) and compounds with 5 carbons or more (including
aromatics - C₅₊) after 1 h on stream of the samples (commercial
CBV3020E and z_500LO of the invention)

| Samples | % S_{C2H4} | % S_{C3H6} | % S_{C4H8} | % S_{C5+} |
|---|---|---|---|---|
| CBV3020E | 3 | 26 | 9 | 30 |
| z_500LO | 15 | 28 | 13 | 32 |

Hence, it is shown that the incorporation of oxidized lignin in ZSM-5 zeolite synthesis is successful. In depth characterization assessed for one of the samples possessing the lowest Si/Al ratio ever reported, maintaining the sole presence of MFI microporous and crystalline structure.

The catalytic performance of this new material led to an increased light olefins selectivity and longer catalyst lifetime, which may lead to potential industrial applications.

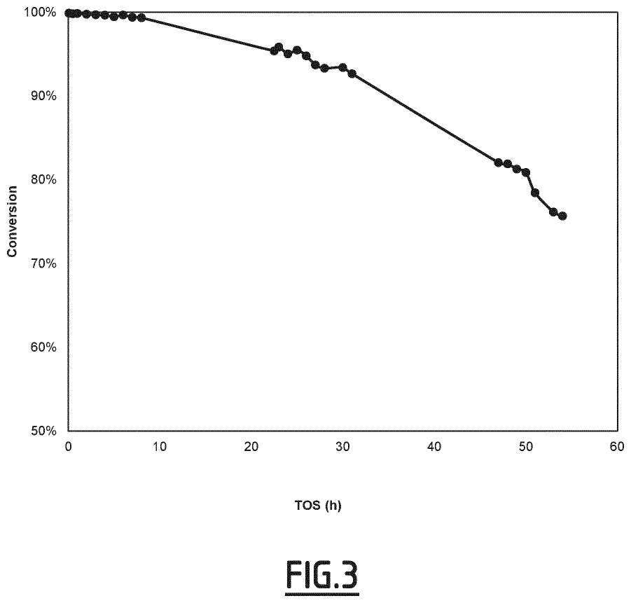

FIG. 3 presents the results of methanol conversion (in %) over z_500L0 obtained at 450° C. and WHSV (weight hourly space velocity) =1.1 h -1 , as a function of time on stream (TOS). Selectivities towards the different products are indicated in Table 6.

Catalytic Performance of z_500LO of the Invention in the Methanol-to-Hydrocarbons Reaction as Compared to Prior Art Zeolites

Surprisingly, the catalytic performance achieved in the methanol conversion into hydrocarbons by z_500LO catalyst according to the invention appears clearly different from the ones obtained with the prior art biomass-templated zeolites, that is:
the prior art ZSM-5 zeolite with a high Si/Al ratio obtained with wood lignin as disclosed in FIG. 5(a) of Pereira et al, 2016 (Influence of Biomass Residues on the Metastability of Zeolite Structures, Nanoscience and Nanotechnology Letters, Vol.8, 1-7, 2016); and
the prior art ZSM-5 zeolite with a Si/Al ratio of 7.6 obtained with washed alkaline hydrolysis of biomass extracted from sugar cane bagasse (zeolite “ZSM-5-a” as disclosed in Pereira et al, 2018, Biomass-mediated ZSM-5 zeolite synthesis: when self-assembly allows to cross the Si/Al lower limit, Chemical Science, 2018, 9, 6532-6539). Said zeolite ZSM-5-a does not contain any wood lignin.

Indeed, the catalyst stability (life-time) is seriously improved with respect to its counterparts despite the presence of numerous acid sites, due to the high Al-loading in the zeolite frame.

In addition, the selectivity towards ethylene and butylenes is favored for the z_500LO catalyst of the invention, typically Methanol-To-Olefins (MTO) behavior, whilst a clear Methanol-To-Gasoline (MTG) was observed for the former prior art zeolites, which showed up to 60% selectivity towards C₅₊ hydrocarbons fraction produced.

The stability of these zeolites submitted to high temperatures, steam presence or further regeneration in air, has been successfully evaluated: the zeolite of the invention (z_500LO) is the most stable.

### Catalytic Performance in the Cracking of N-Hexane (So Called α-Test)

The high cracking rate of n-hexane achieved at 500° C. over the prior art ZSM-5-a zeolite disclosed in Pereira et al, 2018 mentioned above (Biomass-mediated ZSM-5 zeolite synthesis: when self-assembly allows to cross the Si/Al lower limit, Chemical Science, 2018, 9, 6532-6539) was measured according to the protocol described in said publication.

Briefly, n-hexane cracking experiments were performed in a high-throughput unit (Vinci Technologies). Eight tubular quartz reactors assembled in parallel to each other (187 mm in length and 6 mm internal diameter) were used. After cationic exchange (80° C. for 4 h with a 1 M NH₄NO₃ aqueous solution, repeated three-times) and calcination 5 h at 500° C. in air, ZSM-5 zeolite was activated under nitrogen at 500° C. for 2 h prior to catalytic evaluation at the same temperature, then the flow was shifted to an 11% volumic of n-hexane in nitrogen (60 mL/min). The products were injected on line after 3, 17 and 32 min on stream using a GC-2010 Shimadzu chromatograph. The set-up details as well as chromatographic conditions were already reported in A. J. Maia, B. Louis, Y. L. Lam and M. M. Pereira, J. Catal., 2010, 269, 103. The activity was presented as the average result obtained at 3, 17 and 32 min on stream (in the absence of significant deactivation).

The high initial cracking rate of n-hexane at 500° C. over the prior art ZSM-5-a zeolite was equal to 3856 μmol.gcat⁻¹.min⁻¹ with a ratio propylene/propane=0.88.

Preliminary tests performed over the z_500LO zeolite of the invention under the same conditions led to a drastic improvement with respect to commercial ZSM-5 zeolite (Petrobras, Si/Al=25): the initial reaction rate was three-times superior for z_500LO over Petrobras zeolite. The selectivity towards propylene was also superior for z_500LO whatever the degree of conversion (verified up to 40%). Finally, outstanding ratios propylene/propane>2.5 were obtained with z_500LO.

### Example 2: Synthesis of ZSM-5 Zeolites According to the Invention With Different Lignins and Characterization

### 1. Synthesis of ZSM-5 Zeolites With Different Lignins (Si/Al Molar Ratio=10 in the Gel)

Two lignin-assisted ZSM-5 zeolite synthesis recipes were prepared according the hydrothermal method with the following molar ratios:
(i) ZSM-5 type A: NaAlO₂:TEOS:TPAOH:NaCl:H₂O=1:10:13:4.4:3000 (molar ratio TEOS:NaCl of 10:4.4, i.e. around 2.27), or
(ii) ZSM-5 type B: NaAlO₂:TEOS:TPAOH:NaCl:H₂O=1:9.6:14.1:4.3:2307 (molar ratio TEOS:NaCl of 9.6:4.3, i.e. around 2.23).

Specifically for synthesis (i) (ZSM-5 type A), 0.125 g sodium aluminate anhydrous (NaAlO₂) and 0.375g sodium chloride (NaCl) were added in a 500 mL Erlenmeyer flask containing 30 mL of distilled water. Then, 19.1 mL tetrapropylammonium hydroxide (TPAOH, Sigma-Aldrich, 20% in weight in water) was mixed to the solution under stirring. Then, 30 mL of deionized water was added to the solution, followed by the addition of 3.5 mL tetraethylorthosilicate (TEOS, 99%) dropwise to the solution under vigorous stirring (ca. 600 rpm). Finally, 0.3 g lignin powder was poured in the solution. Ageing and homogenization of the mixture were performed during 2 h, at room temperature. The gel was then transferred to a Teflon-lined stainless-steel autoclave (60 mL effective volume) and placed in an oven at 170° C. for 7 days (crystallization).

After the crystallization, the solution was filtered and washed with distilled water until pH 7 and dried at 110° C. overnight. The obtained powder was calcined at 550° C. for 15 h in air to remove the structure directing agent and to obtain Na-ZSM-5 zeolite. The obtained white powder was ion-exchanged three times with 30 mL NH₄NO₃ aqueous solution (1 M) per 0.2 g of ZSM-5 at 80° C. under stirring for 1 h. The solution was then filtered and washed with deionized water followed by drying at 110° C. in an oven. The ammonium zeolite-form was calcined in air at 550° C. for 15 h to produce acidic H-ZSM-5 zeolite.

For synthesis (ii) (ZSM-5 type B), the same steps were used, with the molar ratios indicated for (ii). 0.3 g of lignin powder was also used.

The “lignin powder” indicated in the above synthesis is as defined in section 2 below.

It is worthy to mention here that the two ZSM-5 A and B-types were obtained with different recipes than the one reported by Gomes et al (Microporous and Mesoporous Mater. 254 (2017) 28-36) using lignin originated from the city of Kirov. Indeed, a nearly three-times higher sodium chloride and silica source were used in said former study (in the study of Gomes et al, the molar ratio TEOS:NaCl is 27:13, i.e. around 2.08).

### 2. Characterization of Lionin Samples

### 2.1. Used Lignins

8 lignins were used:

4 types of lignins (lignosulfonates) were provided by Borregaard (Norway) having different molecular weights (MW) and different sulfur contents (S), as follows:
DP-22664: >90% polymer (<10% water); density 650 kg/m³; low MW; low S-content; 50.4 at % C; 4.6 at % H;
DP-22665: >90% polymer; medium MW; high S-content; 38.3 at % C; 4.1 at % H;
DP-22666: >90% polymer (<10% water); density 500-630 kg/m³; high MW; medium S-content; 48.4 at % C; 5.0 at % H; and
DP-22667: >90% polymer (<10% water); density 650 kg/m³; medium MW; medium S-content; 41.9 at % C; 4.6 at % H.

The 4 following lignin sources were also used, and their content was characterized as follows:
Kraft alkali lignin (low sulfonate content, Aldrich, as indicated in Example 1): 46.5 at % C and 4.9 at % H;
walnut shell (eco-shell, as indicated in Example 1): 47.5 at % C ; 6.1 at % H ; 0.2 at % N;
lignin from the Kirov plant (city of Kirov, Russia, as indicated in Example 1; “HL”); and
oxidized lignin (“OHL”) of the lignin from the Kirov plant: 56.9 at % C and 5.1 at % H. The oxidation procedure of the HL lignin for obtaining the OHL lignin is described below.

### 2.2. Oxidation Procedure of the HL Lignin for Obtaining the OHL

There are three possible procedures:

The first procedure for HL oxidation into OHL has been reported by Evstigneyev et al. (E. I. Evstigneyev, O. S.Yuzikhin, A. A.Gurinov, A. Y. Ivanov, T. O. Artamonova, M. A. Khodorkovskiy, E. A.Bessonova, A. V.Vasilyev, J. Wood Chem.Technol.36 (2016) 259).

It comprises an alkali treatment: specifically, HL dissolution was carried out in a 1 L round-bottomed three-necked flask equipped with a thermometer, a propeller stirrer and a reflux condenser, on a mantle heater. The alkali solution (3.6 g of NaOH in 0.5 L of water) was placed in a flask and 20 g of HL were added in small portions under vigorous stirring. The temperature in the flask was then raised to 85° C. and stirring was continued for 1 h. Finally 0.5 L of OHL solution (pH 9.5) was cooled and obtained with a concentration of 40 g/L.

The second procedure comprises a treatment with molecular oxygen and has been adapted from Rahimi et al. (Nature 515 (2014) 249-252): HL has been oxidized into OHL using molecular oxygen and then further treated with a mixture of formic acid and sodium formate. A soluble fraction of low molecular weight soluble aromatics of 61% in weight was obtained, whilst 30% of non-soluble oligomeric species were formed, as described in the scheme hereunder:

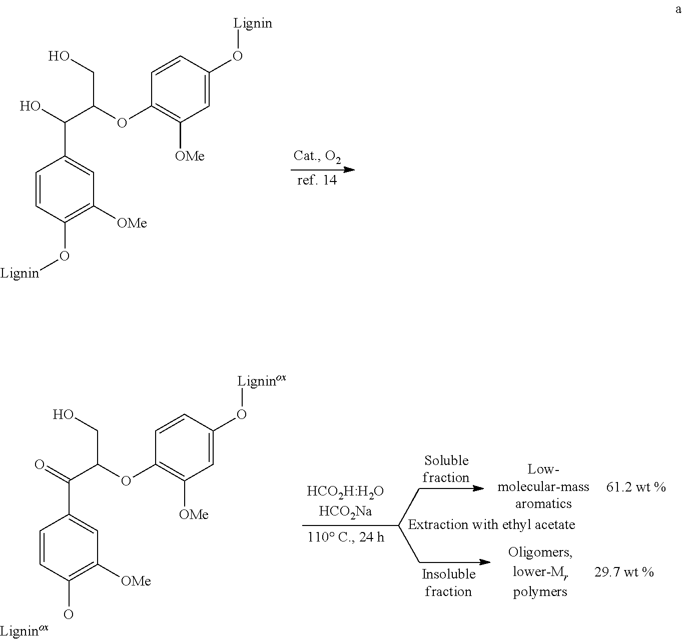

Finally, the third possible procedure for oxidizing HL may be performed by hydrogen peroxide (H₂O₂) in the presence of sulfuric acid to yield OHL.

### 2.3. Compositions of the HL and the OHL

The methods used for determining the composition of HL and OHL samples were the followings:
(i) Klason lignin (non-soluble residue) and acid-soluble lignin were determined according to the methods reported by Dence (C. W. Dence, The determination of lignin. In Methods in Lignin Chemistry; Lin, S. Y.;Dence, C. W., Eds.; Springer-Verlag: Berlin, 1992; 33-61).
(ii) Carbohydrates contents were determined by photocolorimetry using the phenol-sulfuric acid method (E. I. Evstigneyev, Russian J. Bioorg. Chem. 43 (2017) 732).
(iii) The quantity of methoxy groups (determined by the reaction between lignins and hydroiodic acid) and the total ash content were determined according to Zakis (G. F. Zakis, Functional Analysis of Lignins and their Derivatives. Atlanta, GA: TAPPI Press, 1994).
(iv) Carboxyl and phenolic groups were analyzed by conventional methods slightly modified for lignin analysis (E. I. Evstigneyev, Russian J. Appl. Chem. 86 (2013) 258).

The chemical composition and properties of HL and OHL (OHL obtained according to the first procedure described in section 2.2.) are given in Table 7:

TABLE 7
Characterization of HL and OHL (obtained according
to the first procedure), content, mass %

| | Klason | Carbo- | | | | | |
|---|---|---|---|---|---|---|---|
| Lignin | lignin | hydrates | OMe | COOH | OHₚₕₑₙ | C═O | Ash |
| HL | 89.5 (0.3)^{a} | 6.8 | 12.0 | 4.5 | 3.0 | 4.2 | 3.4 |
| OHL | 87.4 (2.7)^{a} | 5.1 | 5.5 | 10.3 | 2.1 | 5.1 | 1.0 |

Note:
^{a}content of acid-soluble lignin is given under brackets

The structure of OHL obtained by the third procedure (H₂O₂) was studied by solid-state ¹³C NMR spectroscopy (data not shown). It is shown that aromatic rings of HL have been oxidized into muconic acid type structures, which correspond to muconic acid structures of formula (II) mentioned in the description.

The successful oxidation procedure is further confirmed by the IR data which assessed the presence of the characteristic C═O unconjugated stretching vibration at 1713 cm⁻¹ only appeared in the oxidized lignin, corresponding to the formation of muconic acid groups on HL aromatic rings.

### 2.4. ZSM-5 Zeolites Nomenclature

The following nomenclature is used:
ZSM-5 zeolites of type A or B (see section 1 (i) and (ii) above) are obtained. They include different lignins (as indicated in section 2.1 above).
ZSM-5 type A obtained with DP-22664 are called “ZSM-5 type A DP-22664”.
ZSM-5 type A obtained with DP-22665 are called “ZSM-5 type A DP-22665”.
ZSM-5 type A obtained with DP-22666 are called “ZSM-5 type A DP-22666”.
ZSM-5 type A obtained with DP-22667 are called “ZSM-5 type A DP-22667”.
ZSM-5 type A obtained with walnut shells are called “ZSM-5 type A walnut shell”.
ZSM-5 type B obtained with OHL (the oxidized lignin of the lignin from the Kirov plant) are called “ZSM-5 type B OHL”. For this zeolite, 0.3 g of OHL (300 mg) was used, but similar experiments were made using 0.1 g (100 mg) or 0.5 g (500 mg) of OHL.

### 3. Characterization of Selected ZSM-5 Zeolite Samples

a) ZSM-5 type A DP-22664 to ZSM-5 type A DP-22667 (which are ZSM-5 with Si/Al molar ratio of 3-4):

The presence of the following elements has been assessed by EDX in calcined and exchanged material in its H-form: Oxygen 74.9 wt %; Sodium 0.1 wt %; Aluminium 6.5 wt %; Silicon 18.5 w t%. This corresponds to a bulk Si/Al=3 for this material prepared according to type A (recipe (i) in section 1, data not shown).

The chemical composition of lignin appears to impact the Si/Al ratio of the obtained ZSM-5 material. To reach the lowest ratio, it is preferred to have a lignin composition with:
high S-content; and
medium Molecular Weight (MW).

In other words, to reach the lowest Si/Al molar ratio, it is preferred to use a lignin composition similar to the one of DP-22665.
b) ZSM-5 type B OHL (which are ZSM-5 with Si/Al molar ratio of 3-8):

EDX mapping confirms a highly homogeneous distribution of Al and Si atoms throughout the crystal. An average Si/Al value of 3.5 could be measured using 500 mg of OHL during the synthesis protocol (see Table 8 below). While diminishing the quantity of OHL to 300 mg and 100 mg, a significant raise in the Si/Al molar ratio to 6 and 8 could be observed, respectively.

TABLE 8
Characterization of ZSM-5 type B OHL (with 500 mg of OHL)

| Element | Weight % | Atomic % | Net Int. | Error % |
|---|---|---|---|---|
| Al content | 9.5 | 6.8 | 185.2 | 6.6 |
| | 9.4 | 6.7 | 180.1 | 6.5 |
| | 8.6 | 6.6 | 348.8 | 4.5 |
| Si content | 31.3 | 21.6 | 500.3 | 6.2 |
| | 30.6 | 21.0 | 482.2 | 6.2 |
| | 38.8 | 28.4 | 1346.5 | 4.5 |

Thus, one can observe that the higher amount of OHL is added, the lowest Si/Al is achieved. Pictures of these 3 zeolites (i.e. with 100 mg, 300 mg or 500 mg of OHL) all show homogenous crystals (data not shown).

For ZSM-5 type B OHL with 500 mg of OHL, in spite of the sole presence of MR structure in the XRD pattern (data not shown), it appears that some peaks cannot be indexed using conventional orthorhombic unit cell (Pnma space group); the latter peaks are super lattice reflections due to Al ordering. A careful inspection of the XRD pattern shows that those extra-reflections appear as shoulders at slightly smaller 2θ angles than the main peaks, thus forming doublets in those peaks. As already reported in Pereira et al (M. M. Pereira, E. S. Gomes, A. V. Silva, A. B. Pinar, M. G. Willinger, S. Shanmugam, C. Chizallet, G. Laugel, P. Losch, B. Louis, Biomass-mediated ZSM-5 zeolite synthesis: when self-assembly allows to cross the Si/Al lower limit, Chem. Sci. 9 (2018) 6532-6539), the presence of a second unit cell with larger dimensions (due to more Al insertion in the framework) could be assessed. Besides, pyridine adsorption measurements confirmed the nearly absence of the vibration at 1455 cm⁻¹ corresponding to Lewis acid sites. The ratio between the bands at 1546 cm⁻¹ (Brønsted acid sites) and 1455 cm⁻¹ (Lewis acid sites) could be estimated to 20, thus confirming the full introduction of Al-atoms within the zeolite frame.
c) ZSM-5 type A walnut shell (which are ZSM-5 with Si/Al molar ratio of 6-7):

The use of walnut shells allowed achieving ZSM-5 zeolite crystal spheroids having approximately a diameter of 6 μm. Numerous nano-sized elongated sub-units form these spheres. According to EDX analysis, a bulk Si/Al=6.6 could be assessed (see Table 9).

TABLE 9
Characterization of ZSM-5 type A walnut shell

| Element | Weight % | Atomic % | Net Int. | Error % |
|---|---|---|---|---|
| C content | 3.3 | 5.4 | 267.0 | 9.9 |
| O content | 52.1 | 63.5 | 10051.1 | 5.1 |
| Al content | 5.7 | 4.1 | 698.5 | 4.9 |
| Si content | 38.9 | 27.0 | 4070.1 | 4.5 |

d) Thermal analysis of ZSM-5 zeolites prepared with different lignins:

Thermogravimetric analysis of non-calcined ZSM-5 samples (i.e. ZSM-5 type A DP-22665 to ZSM-5 type A DP-22667) has been performed to evaluate the organic matter content present within the ZSM-5 zeolites. It clearly appears that a larger weight loss was observed between 400° C. and 450° C. in ZSM-5 zeolites type A DP-22665 to DP-22667, as compared to the reference zeolite prepared in the absence of lignin (data not shown).

This indicates the presence of supplementary organic matter, being between 2% and 4% in weight, originating from the lignosulfonate to the normal presence of TPA⁺ template cations.

### Example 3: Synthesis of ZSM-5 Zeolites With Different Ligins Having a Si/Al=14 in the Gel According to the Invention

The ZSM-5 zeolite was synthesized by using the following initial composition of the gel:
(iii) 1 NaAlO_{2:}13 NaCl:14 TEOS:3 TPAOH:3718 H₂O.

Sodium chloride (0.760 g, Janssen Chimica, P. A.), tetrapropylammonium hydroxide (TPAOH, 6.0 g, Sigma-Aldrich, 1 M in H₂O), sodium aluminate (NaAlO_{2,} 0.080 g, Sigma-Aldrich) and distilled water were mixed until a clear solution was obtained. Then, TEOS 2.8 g, Sigma-Aldrich, 99%) and lignin DP-22665 (mentioned in part 2 of Example 2) (0.6 g) were added to the solution. After, the synthesis gel was aged for 1.5 h at room temperature under stirring. The synthesis gel was set inside a Teflon-lined autoclave (40 mL) and the zeolite crystallization performed under static condition at 170° C. for 24 h. After cooling down, the solid was recovered by filtration and washed until neutral pH. The final solid was calcined at 600° C. for 5 h under air.

In order to obtain the zeolite acid form, two successive exchanges using 2 M NH₄NO₃ aqueous solution at 80° C. for 1 h (1g of zeolite per 50 mL of solution) were performed. The ammonium form was converted into the protonic form by calcination at 450° C. for 4 h under air.

The same procedure has been conducted but the quantity of TPAOH was reduced down to 1, according to (iv) 1 NaAlO_{2:}13 NaCl:14 TEOS:1 TPAOH:3718 H₂O. It has been verified by XRD (not shown) that a crystalline ZSM-5 pure structure could still be obtained while reducing the quantity of organic template (TPAOH) by 20% to 80%, preferably between 60% and 70%. The presence of lignin may compensate the presence of TPAOH at least to some extent.

The mass ratio between lignin and NaAlO₂ (mass ratio lignin/NaAlO₂) was varied between 2 and 15 in the synthesis recipe, preferably between 3 and 8.

ZSM-5 zeolites according to the invention were obtained, which have in both cases (i.e. (iii) and (iv)) a Si/Al ratio of 6±1.

1. Process for preparing a zeolite ZSM-5 exhibiting a Si/Al molar ratio comprised between 2 and 8, preferably between 3 and 8, comprising the following steps:
a) mixing at least one silicon source, at least one aluminum source, at least one organic template and at least one aqueous solvent, in order to obtain a synthesis mixture in solution or gel form;
b) ageing the mixture obtained in step a) at a temperature of between 20° C. and 200° C. during at least 30 minutes; and
d) crystallizing the resulting mixture during at least 24 hours, wherein a step c) of adding wood lignin or oxidized wood lignin to the mixture is performed after step a) or after step b).

2. Process according to claim 1, wherein it further comprises a step e) of separating the solid obtained in step d) by means of centrifugation, filtration or evaporation of the solvent.

3. Process according to claim 1, wherein it comprises the following steps:
a) mixing at least one silicon source, at least one aluminum source, at least one organic template and at least one aqueous solvent, in order to obtain a synthesis mixture in solution or gel form;
b) ageing the mixture obtained in step a) at a temperature of between 20° C. and 200° C. during at least 30 minutes;
c) adding wood lignin or oxidized wood lignin to the mixture of step b) ; and
d) crystallizing the mixture of step c) during at least 24 hours.

4. Process according to claim 1, wherein it comprises the following steps:
a) mixing at least one silicon source, at least one aluminum source, at least one organic template and at least one aqueous solvent, in order to obtain a synthesis mixture in solution or gel form;
c) adding wood lignin or oxidized wood lignin to the mixture of step a);
b) ageing the mixture obtained in step c) at a temperature of between 20° C. and 200° C. during at least 30 minutes; and
d) crystallizing the mixture of step b) during at least 24 hours.

5. Process according to claim 1, wherein the silicon source is chosen from tetraethylorthosilicate (TEOS) (C₈H₂₀O₄Si), colloidal silica, disodium metasilicate (Na₂O₃Si) and their mixtures.

6. Process according to claim 1, wherein the aluminum source is chosen from sodium aluminate (NaAlO₂), aluminum isopropoxide (C₉H₂₁AlO₃), aluminum sulfate (Al₂O₁₂S₃) and their mixtures, and/or wherein the organic template is chosen from tetrapropyl ammonium hydroxide (TPAOH) (C₁₂H₂₉NO), tetramethyl ammonium hydroxide (TMAOH) (C₄H₁₃NO), tetramethyl ammonium bromide (C₄H₁₂BrN), tetrapropyl ammonium bromide (C₁₂H₂₈BrN) and their mixtures.

7. Process according to claim 1, wherein step a) comprises the following sub-steps:
a1) mixing at least one aluminum source with at least one organic template in at least one aqueous solvent, in order to obtain a mixture;
a2) adding at least one silicon source, and preferably at least one salt, more preferably at least sodium chloride, into the mixture of step a1), in order to obtain the synthesis mixture in solution or gel form,
preferably the molar ratio of the silicon source to the salt, preferably sodium chloride, is of at least 2.20, preferably between 2.20 and 3, preferably between 2.20 and 2.80, preferably between 2.20 and 2.50.

8. Process according to claim 1, wherein the wood lignin presents at least one, preferably all, of the following features:
it presents an ash content of between 3% to 10%, preferably from 5% to 8% by weight of the total weight of the dry wood lignin;
it presents a residual carbohydrate content of between 5% to 20%, preferably from 8% to 20%, preferably from 10% to 15% by weight of the total weight of the wood lignin;
some parts of the wood lignin are extracted in at least one organic solvent, which may be polar or apolar, preferably the organic solvent is chosen from hexane, chloroform and acetone, wherein an amount of 3% to 10% by weight of the total weight of the wood lignin is extracted in hexane, preferably an amount of 4% to 8% by weight; an amount of 3% to 10% by weight of the total weight of the wood lignin is extracted in chloroform, preferably an amount of 4% to 8% by weight; and an amount of 1% to 10% by weight of the total weight of the wood lignin is extracted in acetone, preferably an amount of 1.5% to 5% by weight; and/or
it presents a Klason lignin content of between 50% to 95%, preferably from 50% to 90%, preferably from 50% to 80%, preferably from 60% to 70% by weight of the total weight of the wood lignin; and/or
it presents a sulfur atomic content of between 0.8 at % to 8 at %, preferably from 3 at % to 6 at % by weight of the total weight of the dry wood lignin
it presents a carbon atomic content of between 35 at % to 55 at %, preferably from 37 at % to 51 at % by weight of the total weight of the dry wood lignin; and/or
it presents a hydrogen atomic content of between 3.8 at % to 6.5 at %, preferably from 4 at % to 6.2 at % by weight of the total weight of the dry wood lignin; and/or
it comprises a nitrogen atomic content of between 0.1 at % to 0.5 at % by weight of the total weight of the dry wood lignin.

9. Process according to claim 1, wherein the wood lignin is oxidized and is obtained by a chemical treatment, preferably an alkali treatment of the wood lignin, preferably the oxidation is performed by mixing the wood lignin with an alkali solution, and may be heated, typically at a temperature of 70° C. to 100° C., preferably of 80° C. to 90° C., typically for at least 1 h.

10. Process according to claim 1, wherein the ageing of step b) is performed during at least 1 h, preferably at least 1 h30, preferably between 1 h and 5 h.

11. Process according to claim 1, wherein the zeolite ZSM-5 presents a Si/Al molar ratio comprised between 3 and 7.6, preferably between 3 and 7, preferably between 3 and 4.

12. Zeolite ZSM-5 presenting a Si/Al molar ratio comprised between 2 and 8, preferably between 3 and 8, which is obtainable by the process according to claim 1.

13. Zeolite ZSM-5 according to claim 12, which presents:
a specific surface area of between 150 m²/g and 250 m²/g, preferably between 180 m²/g and 220 m²/g, and
a total pore volume of between 0.01 cm³/g and 0.5 cm³/g, preferably between 0.05 cm³/g and 0.2 cm³/g, wherein the microporous volume is 70% of the total pore volume, as measured by N₂ adsorption-desorption.

14. Zeolite ZSM-5 according to claim 12, wherein it is in the form of crystals presenting an oblong shape with a length and a width, the length being greater than the width, for example at least 2 times greater; preferably the crystals have a length of from 10 μm to 30 μm, preferably of from 15 μm to 25 μm.

15. Use of a zeolite ZSM-5 according to claim 12, in at least one of the following applications:
as a catalyst, especially in hydrocarbon conversions, preferably Fluid Catalytic Cracking such as methanol to olefins conversion or n-hexane cracking;
for adsorbing or desorbing liquids and/or gases, such as water treatment or industrial gases treatment;
for selective separations of gas or liquid mixtures, i.e. as molecular sieves;
as detergents;
for treating pesticides, organic chlorine or hydrocarbons-loaded effluents;
for remediating heavy metals contained in soils or waters;
for purifying soils or waters from radioactive elements, such as cesium; or
as a seed in an industrial process for preparing ZSM-5 zeolites presenting a Si/Al molar ratio comprised between 2 and 8, preferably between 3 and 8.
